(19) United States
(12) Patent Application Publication
(10) Pub. No.: US 2020/0030744 A1
(43) Pub. Date: Jan. 30, 2020

(54) AGENT FOR REMOVING HALOGEN GAS, METHOD FOR PRODUCING SAME, METHOD FOR MONITORING THE CONSUMPTION STATE OF THE REMOVAL AGENT

(71) Applicant: CLARIANT CATALYSTS (JAPAN) K.K., Tokyo (JP)

(72) Inventors: Kenichiro Sunata, Toyama (JP); Tadahito Nakashima, Toyama (JP); Hyun-Joong Kim, Toyama (JP)

(21) Appl. No.: 16/512,900

(22) Filed: Jul. 16, 2019

(30) Foreign Application Priority Data
Jul. 30, 2018 (JP) 2018-142248

(51) Int. Cl. B01D 53/68; G01N 33/00; B01D 53/30; B01D 53/73

(52) U.S. Cl. CPC B01D 53/685; G01N 33/0052; B01D 53/30; B01D 2251/602; B01D 2253/104; B01D 2257/20; B01D 2251/608; B01D 53/73

(57) ABSTRACT

A halogen gas removing agent for removing halogen gas from a gas flow, which reduces the risk of leakage of the halogen gas exhausted from a semiconductor production process by treating the gas flow with the removing agent and a process for producing the removing agent. Also provided are an apparatus for removing the halogen gas using the removing agent, and a method for monitoring the state of consumption of the halogen gas. The halogen gas removing agent includes an inorganic compound base material, a sulfur-containing reducing compound and a color indicator, preferably using a pseudoboehmite as the base material, adding a pH indicator having a transition range in a pH range of 3 to 8 as the color indicator, and adding a basic metal compound such as zinc oxide.

### BACKGROUND OF THE INVENTION

### Field of the Invention

The present invention relates to a halogen gas removing agent which is capable of efficiently decomposing and removing a halogen-based gas, particularly a halogen-based waste gas arisen from halogen used in/as an etching gas or a cleaning agent in the production process for semiconductors, and the state of the consumption of which, can be monitored by a simple and reliable method to enable prediction of a residual life of the removing agent and to enable the reduction of the risk of leakage of a toxic gas therefrom.

### Description of the Related Art

Examples of halogen-based gases include F₂, Cl₂, Br₂, ClF₃, BrF₃ and BrF₅, and in a broad sense, also include many kinds of halogenated non-metallic gases such as SiF₄ and BCl₃. As one of the methods for removing these halogen-based gases, a method in which a halogen gas is physically adsorbed on a porous body such as carbon black is conventionally known. This method is a low capacity method, and when the used adsorbent is replaced with a new one, there is a risk that a harmful gas is freed to adversely affect the environment. As an alternative method, a scrubber method in which a halogen gas such as Cl₂ is brought into contact with water, thereby changed into hydrogen chloride, absorbed and then neutralized with an alkali such as caustic soda is known. According to this method, treatment of a large amount of a halogen-based gas becomes possible, but complicated operations such as preparation and management of a solution for the treatment, and treatment of waste liquid are needed. On that account, instead of the above methods, a dry removing method using a solid treating agent that is easy to handle has been spreading in recent years.

Typical performances required of a dry treating agent for a halogen-based gas are as follows.
(1) A high ability to treat a halogen-based gas per unit weight of the treating agent.
(2) Ability of the removing agent to fix a harmful gas therein and, when the used removing agent is replaced with a new one, absence of liberation/diffusion of the harmful gas, and easy replacement and disposal of the removing agent.
(3) When the removing agent is used, the life of the removing agent is predictable by monitoring the state of consumption of the removing agent.

The above (1) is very important particularly in the production process for semiconductors wherein a large amount of a halogen-based gas is consumed. In order to achieve this, a proposal for a removing agent containing an inorganic compound base material such as an oxide or a hydroxide of a solid metal and a reducing agent is made in, for example, JP2001017831A. By virtue of this technique, item (1) has been greatly improved. However, as it cannot yet satisfy the needs for a semiconductor production process that has been increased in scale, further improvements are required.

Monitoring the state of consumption of the removing agent and thereby predicting the life of the removing agent, as mentioned in (3) above, and as required of the halogen removing agent are important techniques for avoiding a serious risk of leakage of a toxic gas. In order to enable this, not only is a function to detect a leaked toxic gas needed but also the state of consumption of the removing agent needs to be quantitatively detected to thereby enable the prediction of a residual life of the removing agent before the breakthrough of the toxic gas.

As an attempt close to that, JP3567058B describes a halogen gas detection agent obtained by adding Congo red as a color indicator to a hydroxide of a transition metal. The agent has a function of detecting a halogen gas by the color change reaction of Congo red, but the halogen gas removing ability of the agent is low and, in order to carry out the removal of halogen, it is necessary to connect a halogen removing agent before the detection agent. Even in this case, however, it is impossible to monitor the state of consumption of the removing agent and to predict the breakthrough time even if it can be detected that a toxic gas has broken through the removing agent.

JPH0716582B states, as another example in which halogen gas can be detected, that the removal of halogen and detection of an acidic gas are made possible by packing asbestos comprising caustic soda supported thereon at a column inlet side, an adsorbent comprising basic dye supported on a silica gel at its downstream side, and a basic ion-exchange resin at the end. By this method, the time required for halogen to reach the detection agent packed portion from the inlet can be detected, but it is difficult to monitor the state of consumption of the removing agent and predict the residual life of the removing agent before the breakthrough of the gas.

In order to solve items (1) and (2) above of the conventional problems, the present applicant has filed a patent application directed to a halogen gas removing agent that has been remarkably improved in terms of performance for treating the halogen gas by using pseudoboehmite and a sulfur-containing reducing compound as main components and by further simultaneously using, if necessary, a basic metal compound such as zinc oxide (Japanese Patent Application No 2017-020456).

The removing agent in Japanese Patent Application No 2017-020456 has an extremely high ability to treat a halogen gas, but the state of consumption of the removing agent cannot be monitored. The sulfur-containing reducing compound used to increase the removing ability increases the ability to remove halogen, but on the other hand, sulfurous acid gas is formed as a by-product, and the risk that this gas passes through the removing agent and leaks out is brought about (particularly in cases where the basic metal compound is not simultaneously used). From such a viewpoint, it is desirable for the removing agent to be able to carry out not only monitoring of a halogen gas and a hydrogen halide but also monitoring of sulfurous acid gas.

In the actual circumstances, improvements in the removing ability of the solid, dry type treating agent have been observed as described above, but no proposal of a technique to satisfy all the requirements (1) to (3) has been made.

### SUMMARY OF THE INVENTION

It is a first object of the present invention to provide a halogen gas removing agent capable of efficiently decomposing a halogen-based gas, particularly a halogen-based waste gas arisen from halogen used in/as an etching gas or a cleaning agent in the production process for semiconductors.

It is a second object of the present invention to provide a method and a system both of which can accurately predict the residual life of the removing agent and prevent external leakage of a harmful gas by monitoring the state of the consumption of the removing agent.

It is a third object of the present invention to provide a system that detects the presence of not only a hydrogen halide formed by decomposition of a halogen gas but also a sulfurous acid gas that may be formed at the same time, in the removing agent, and prevents leakage thereof.

It is a fourth object of the present invention to make it possible to use the removing agent to the extent close to the ability limit of the removing agent by monitoring the state of the consumption of the removing agent, thereby decreasing the frequency of replacement of the removing agent and reducing the running cost.

Further objects of the present invention will be apparent from the following description.

In light of the above circumstances, the present inventors have thoroughly researched in order to overcome the disadvantages of the conventional techniques. As a result, they have obtained the knowledge and guideline below as ideas to achieve the objects of the present invention.

(1) In the conventional method for detecting a halogen gas, by arranging a detection agent behind a halogen removing agent or arranging a detection agent different from a removing agent in series in the middle of a removing agent column, the arrival of a halogen gas is detected. In this method, continuous follow-up observation of the amount of the consumption of the removing agent cannot be carried out, and therefore, prediction of the life of the removing agent lacks accuracy, and risks of breakthrough and external leakage are liable to occur.

(2) Under such circumstances, a method in which a removing agent and a detection agent are not connected in series and a function to detect a decomposed gas is imparted to the removing agent itself has been studied.

(3) As a first function therefor, a function to detect hydrogen chloride that is a decomposed gas is necessary. Secondly, in order to monitor the state of consumption of the removing agent, it is necessary that a color indicator should not significantly lower the removal performance of a removing agent when the removing agent and the color indicator are mixed and used, and thirdly, in order that a trace amount of a harmful gas can be detected, the color indicator needs to have detection ability with high sensitivity. As a material which satisfies the above, a pH indicator, which is also referred to as an acid-base indicator and which gives a sensitive color change even by the addition thereof in a small amount, has been selected.

(4) On the other hand, considering the function of the removing agent from the viewpoint of monitoring the state of consumption of the removing agent, it is preferable firstly that a strongly acidic or basic substance should not be contained in the removing agent. For example, if a strongly basic substance such as slaked lime whose saturated aqueous solution has a pH exceeding 12 is contained in the removing agent, the removing agent becomes strongly basic, and even if a change of pH occurs by the decomposition of a halogen gas, said change of pH is small, and the detection sensitivity is lowered. Likewise, if a strongly acidic substance such as sulfuric acid is contained, the same applies thereto. From that viewpoint, the removing agent (except for the pH indicator contained therein) is preferably neutral to weakly basic because, in this case, a change of pH accompanying halogen decomposition can be detected with high sensitivity. From such a viewpoint, it is particularly preferable to use pseudoboehmite shown in Japanese Patent Application No 2017-020456 as a base material of the removing agent.

(5) With regard to the function of the removing agent seen from the monitoring function, the diffusion rate of the strongly toxic halogen gas in the removing agent is preferably lower than that of the hydrogen halide. The reason for this is that, in the case where decomposition of the halogen gas proceeds in the removing agent and then the hydrogen halide reaches the column outlet side, if the halogen gas having a more serious risk does not reach the column outlet before the time of arrival of the hydrogen halide at the outlet, the safety can be increased as much as possible even if leakage of the hydrogen halide from the column occurs. For the same reason, the diffusion rate of sulfurous acid gas that may be formed by the decomposition of the reducing agent is preferably higher than that of the halogen gas.

(6) As a result of studying halogen removing agents from such a viewpoint, it has been found that a removing agent in which the reducing agent described in JP2001017831A or Japanese Patent Application No 2017-020456, especially the reducing agent described in Japanese Patent Application No 2017-020456, is used for the halogen decomposition is particularly preferable. The reason for this is thought to be that, since the reducing agent, particularly a sulfur-containing reducing compound having water of hydration, can markedly increase the halogen decomposition rate, diffusion of the halogen gas to the column outlet side is delayed, and the hydrogen halide easily diffuses to the column outlet side.

(7) Moreover, in order that both the automatic monitoring due to a color sensing system using a photodiode, etc. and visual monitoring conducted by an operator on a daily basis can be carried out, the selection of the type of a pH indicator having high visibility and optimization of a preferred amount of the pH indicator added in the removing agent have been carried out, and then the present invention has been accomplished.

The present invention relates to the following.

1. A halogen gas removing agent comprising at least an inorganic compound, a sulfur-containing reducing compound and a color indicator.

2. The halogen gas removing agent according to item 1 above, wherein the halogen gas comprises at least one selected from the group consisting of fluorine (F₂), chlorine (Cl₂), bromine (Br₂) and iodine (I₂).

3. The halogen gas removing agent according to item 1 or 2 above, for removing a halogen gas from a gas flow.

4. The halogen gas removing agent according to any one of items 1 to 3 above, wherein the gas flow is a gas flow exhausted from a semiconductor production process.

5. The halogen gas removing agent according to any one of items 1 to 4 above, wherein the inorganic compound is selected from the group consisting of metal oxides, metal hydroxides and metal carbonates.

6. The halogen gas removing agent according to item 5 above, wherein the inorganic compound is an alumina-based compound.

7. The halogen gas removing agent according to item 6 above, wherein the inorganic compound is pseudoboehmite and/or montmorillonite.

8. The halogen gas removing agent according to item 6 or 7 above, wherein the inorganic compound has a specific surface area of 100 m²/g to 500 m²/g.

9. The halogen gas removing agent according to item 8 above, wherein the inorganic compound has a specific surface area of 200 m²/g to 400 m²/g.

10. The halogen gas removing agent according to any one of items 1 to 9 above, further comprising a basic metal compound.

11. The halogen gas removing agent according to item 10 above, wherein the basic metal compound is at least one zinc compound selected from the group consisting of zinc carbonate and zinc oxide.

12. The halogen gas removing agent according to any one of items 1 to 11 above, wherein the sulfur-containing reducing compound is at least one compound selected from the group consisting of thiosulfates, sulfites, dithionites and tetrathionates.

13. The halogen gas removing agent according to item 12 above, wherein the thiosulfate is at least one compound selected from the group consisting of sodium thiosulfate, potassium thiosulfate and ammonium thiosulfate.

14. The halogen gas removing agent according to any one of items 1 to 13 above, wherein the sulfur-containing reducing compound comprises water of hydration.

15. The halogen gas removing agent according to any one of items 1 to 14 above, wherein the color indicator is a pH indicator having a transition range in a pH range of 2 to 9.

16. The halogen gas removing agent according to item 15 above, wherein the color indicator is a pH indicator having a transition range in a pH range of 3 to 8.

17. The halogen gas removing agent according to item 16 above, wherein the pH indicator is at least one pH indicator selected from the group consisting of bromophenol blue, methyl orange and bromothymol blue.

18. The halogen gas removing agent according to any one of items 10 to 17 above, wherein the compositional ratio by weight among the color indicator, the inorganic compound, the sulfur-containing reducing compound and the basic metal compound is 0.001 to 1.0:30.00 to 97.00:1.00 to 40.00:0.00 to 40.00 when the total of these components is 100.

19. The halogen gas removing agent according to item 18 above, wherein the compositional ratio by weight among the color indicator, the inorganic compound, the sulfur-containing reducing compound and the basic metal compound is 0.05 to 0.5:50.00 to 75.00:10.00 to 30.00:10.00 to 30.00 when the total of these components is 100.

20. The halogen gas removing agent according to any one of items 10 to 19 above, wherein the total weight of the color indicator, the inorganic compound, the sulfur-containing reducing compound and the basic metal compound is 90 to 100% by weight, based on the total weight of the removing agent.

21. A method for producing the halogen gas removing agent according to any one of items 1 to 20 above, comprising mixing and/or kneading the color indicator, the inorganic compound, the sulfur-containing reducing compound and optionally the basic metal compound, optionally together with a dispersion medium, and then shaping the mixture, followed by drying.

22. A halogen gas removing apparatus, comprising a container, and a window and/or a color sensor, said window and/or said color sensor being provided in the container, wherein
the container comprises a gas flow inlet and a gas flow outlet,
the container comprises the removing agent according to any one of items 1 to 20 above packed in said container, and
the window and/or the color sensor are adapted for observation and/or detection of a color change of the removing agent accompanying the removal of the halogen gas.

23. A method for monitoring the state of consumption of the halogen gas removing agent, using the apparatus according to item 22 above, by measuring the length of a color-changed portion in the removing agent, from the halogen gas inflow end of the removing agent.

24. A method for removing a halogen gas from a halogen-containing gas, comprising bringing the halogen-containing gas into contact with the removing agent according to any one of items 1 to 20 above, wherein the halogen gas is removed while the state of consumption of the removing agent is monitored by observing and/or detecting a color change of the removing agent accompanying the removal of the halogen gas.

### Advantageous Effects of Invention

According to the present invention:

(1) By combining a neutral to weakly basic inorganic compound base material having high ability to decompose/treat a halogen gas with an indicator exhibiting color reaction by an acid, the state of consumption of the removing agent caused by the decomposition of halogen can be monitored with high sensitivity.

(2) Owing to the effect described in (1), the state of consumption of the halogen gas removing agent can be observed in real time, and therefore, the residual life of the removing agent can be accurately predicted. As a result, serious trouble caused by breakthrough of a harmful gas is easily prevented.

(3) Detection can be carried out visually with high sensitivity, and the object can be achieved by adding a small amount of an indicator, so that the removing ability of the removing agent is not lowered.

(4) The residual removing ability of the removing agent can be easily evaluated, and it becomes possible to use the removing agent to the extent close to the ability limit of the removing agent. By virtue of this, the cost for consumables can be reduced, and the frequency of replacement of columns can be decreased.

(5) Since breakthrough of a halogen-based gas can be detected with the color change of the removing agent, it becomes possible to decrease or eliminate the number of gas detectors conventionally arranged behind the removing agent, thereby reducing equipment costs and maintenance costs.

### BRIEF DESCRIPTION OF THE DRAWINGS

FIG. 1 shows an example of a system for removing halogen gas and a monitoring system according to the present invention (constitution of improved system for removing halogen gas);

FIG. 2 shows changes in reflection spectra in Example 4 according to the present invention before and after chlorine removing treatment (diffuse reflection spectra of the removing agent sample of Example 4 before and after color tone evaluation); and

FIG. 3 shows changes in reflection spectra in Comparative Example 2 before and after chlorine removing treatment (diffuse reflection spectra of the removing agent sample of Comparative Example 2 before and after color tone evaluation).

### DETAILED DESCRIPTION

The present invention is a halogen gas removing agent (hereinafter also referred to as a “halogen gas treating agent”) for removing a halogen gas from a gas flow exhausted from e.g. a semiconductor production apparatus and comprises at least an inorganic compound, a color indicator and a sulfur-containing reducing compound. The removing agent immobilizes a halogen gas therein, or decomposes a halogen gas and immobilizes the resulting decomposition product(s) therein, and therefore, by treating a halogen gas-containing gas with the removing agent, the halogen gas can be removed from the above gas.

In the present invention, the halogen gas is not particularly limited as long as it is a gas containing a halogen element. Examples of the halogen gases include fluorine (F₂), chlorine (Cl₂), bromine (Br₂) and iodine (I₂) that are formed by bonding of halogen elements, and gaseous non-metallic halogen compounds, such as halogen trifluoride (ClF₃), bromine trifluoride (BrF₃), bromine pentafluoride (BrF₅), SiF₄ and BCl₃.

The hydrogen halide in the present invention refers to a compound in which halogen arisen by the decomposition of the halogen gas is bonded to a hydrogen atom, and specific examples thereof include hydrogen chloride gas generated from chlorine gas and hydrogen bromide generated from bromine gas.

Hereinafter, in order to make it easier to understand, descriptions will be made mainly taking chlorine as an example of halogens, and unless otherwise stated, a hydrogen halide is described as hydrogen chloride, and a halogen (in cases where the term relating to halogen is not preceded by the term “hydrogen”) is described as chlorine. These descriptions are also applicable to other embodiments related to halogens other than chlorine, and a person skilled in the art will also properly understand other embodiments by referring to these descriptions.

Hereinafter, a halogen gas and a hydrogen halide may be collectively referred to as a halogen-based gas, and a gas and/or a gas flow containing such a halogen-based gas may be referred to as a halogen-containing gas.

The halogen gas removing agent according to the present invention preferably comprises, as its base material, an inorganic compound. The inorganic compound is hereinafter also referred to as an “inorganic compound base material”.

As the inorganic compound base materials, for example, oxides of alkali metals, alkaline earth metals and transition metals, derivatives thereof, or carbonates of alkali metals, alkaline earth metals and transition metals are used. Of these, the materials described in JP2001017831A, for example, an oxide of at least one metal selected from alkaline earth metals, Fe, Co, Ni, Zn, Mn and Cu(I), can be used.

Further, aluminum compounds, such as oxides, hydroxides or carbonates of aluminum, can also be used as the inorganic compound base material.

The inorganic compound base material in the removing agent is required to have many functions. First, the inorganic compound base material needs to have high physical stability in its surface structure, etc. so that the reaction of chlorine gas with the reducing agent can be maintained even in the presence of chlorine gas, and needs to have a large specific surface area in order to enhance the removing ability per unit weight of the removing agent. Furthermore, the inorganic compound base material needs to have appropriate acidity or basicity so that the pH indicator can sensitively change its color as an acid is generated, as previously described. For such reasons, alumina-based compounds or montmorillonite, etc., is preferable for achieving the objects of the present invention because the pH of its saturated aqueous solution is neutral to weakly alkaline.

In the present invention, the alumina-based compound refers to a compound comprising alumina or alumina hydrate as a main component. Examples of the alumina-based compounds that can be used as the inorganic compound base materials include alumina (Al₂O₃) (α-alumina, γ-alumina, η-alumina, γ-alumina, κ-alumina, θ-alumina, χ-alumina, etc.), gibbsite (Al₂O₃·3H₂O), bayerite, boehmite (AlO(OH)) and pseudoboehmite. Of these, pseudoboehmite is particularly preferable as the inorganic compound base material in the present invention.

The pseudoboehmite in the present invention is an aluminum compound represented by a molecular formula of Al₂O₃·nH₂O (n=1 to 2), and has a structure of two stacked layers of edge-sharing AlO₆ octahedra (octahyrora sheet), said layers being held by hydrogen bonds between the surface aluminol groups. If the pseudoboehmite is heated, it is stable at a temperature up to about 300° C., but at 400° C. or higher, it is dehydrated and becomes y-alumina.

For example, as the pseudoboehmite in the present invention, pseudoboehmite in the form of a powder or an aqueous dispersion (sol) is available (e.g. WISH 6006, Wish Chemicals Yueyang Co., Ltd.), and both can be used in the present invention.

The inorganic compound base material, e.g. the pseudoboehmite particle, in the present invention preferably has a specific surface area of 100 m²/g to 1000 m²/g, more preferably 100 to 650 m²/g, still more preferably 150 to 450 m²/g, and particularly preferably 200 to 400 m²/g. If the specific surface area is smaller than the above values, the reaction rate of chlorine gas with the reducing agent decreases, and the chlorine removing performance is liable to decrease. If the specific surface area is larger than the above values, the physical strength of the inorganic compound base material, for example, pseudoboehmite, decreases and it is difficult to maintain the porous structure thereof, so that the chlorine removing performance is likewise liable to decrease. The specific surface area can be measured by the BET method.

In the present invention, the inorganic compound base material, for example the pseudoboehmite, preferably has a total pore volume of pores having diameters of 3 to 500 nm, of 0.02 ml/g to 2.0 ml/g, more preferably 0.05 ml/g to 1 ml/g, and particularly preferably 0.11 ml/g to 0.7 ml/g, for example, 0.2 ml/g to 0.5 ml/g. The inorganic compound base material, for example the pseudoboehmite, preferably has a total pore volume of pores having diameters of 10 to 500 nm, of 0.002 ml/g to 2.0 ml/g, more preferably 0.005 ml/g to 1 ml/g, and particularly preferably 0.01 ml/g to 0.7 ml/g, for example, 0.02 ml/g to 0.5 ml/g. The total pore volume of pores having diameters of 10 nm to 500 nm is preferably 10% or more, more preferably 25% or more, and still more preferably 40% or more, for example, 60% or more or 70% or more, relative to the total pore volume of pores having diameters of 3.0 nm to 500 nm. The upper limit is not particularly limited, but it can be, for example, 90% or less or 85% or less. Although the reason why such a range of the pore volume is preferable is not clear, it is presumed as follows. In the above range, the sulfur-containing reducing compound such as a thiosulfate for assisting the decomposition of chlorine gas, can be sufficiently supported on the inorganic compound base material, and/or a sufficient contact area of chlorine gas with the inorganic compound base material such as pseudoboehmite can be ensured, so that high removing performance can be achieved. Moreover, when the total pore volume is in the above range, it can be avoided that the removing agent is decreased in physical strength and thereby broken by, for example, the pressure inside the column during use to hinder the introduction of the chlorine gas; and therefore, the decomposition rate can be maintained. The total pore volume can be measured by, for example, the mercury porosimetry.

The content of the inorganic compound base material in the removing agent can be, for example, 30% by weight or more, and preferably 40% by weight or more, based on the total weight of the removing agent. In an embodiment of the present invention, the content of the inorganic compound base material, e.g. pseudoboehmite, is 30 to 97% by weight, preferably 45 to 90% by weight, and particularly preferably 50 to 85% by weight, for example, 55 to 80% by weight, based on the total weight of the removing agent. When the amount of the inorganic compound base material such as pseudoboehmite is in the above range, it is possible to achieve particularly good chlorine decomposition activity.

The sulfur-containing reducing compound (hereinafter also referred to as a “sulfur-containing reducing agent” or a “reducing agent”) in the present invention is not particularly limited as long as it is a reducing compound (reducing agent) having a sulfur atom. For example, thiosulfates, sulfites, dithionites or tetrathionates can be used. For example, when a thiosulfate is used as the sulfur-containing reducing compound, examples of the thiosulfates include sodium thiosulfate, potassium thiosulfate and ammonium thiosulfate. It is preferable to particularly use, as the sulfur-containing reducing compound, a reducing agent comprising water of hydration, such as the aforesaid salts in the form of a hydrate (a salt hydrate). Among them, a pentahydrate of thiosulfate, for example, sodium thiosulfate pentahydrate, is particularly preferable.

The content of the sulfur-containing reducing compound in the removing agent is, for example, 1% by weight to 70% by weight, preferably 5% by weight to 55% by weight, more preferably 10% by weight to 50% by weight, still more preferably 12% by weight to 40% by weight, and particularly preferably 15% by weight to 35% by weight, for example, 15% by weight to 30% by weight, based on the total weight of the inorganic compound base material and the reducing agent. When the amount of the sulfur-containing reducing compound is in the above range, it is possible to achieve particularly good chlorine decomposition activity. When the reducing agent is a salt hydrate, the content and ratio of the reducing agent shown herein are those calculated by including the water of hydration, unless otherwise stated.

The removing agent of the present invention can comprise the reducing agent preferably in an amount of 0.5 to 10% by weight, and more preferably 1 to 8% by weight, for example, 3 to 7% by weight or 4 to 6% by weight, based on the content of the sulfur element, relative to the total weight of the inorganic compound base material and the reducing agent. The sulfur atom content can be measured by combustion in oxygen flow-infrared absorption method.

Additives other than the inorganic compound base material and the reducing agent can be added to the removing agent, when needed. Such an additive is preferably at least one basic metal compound selected from the group consisting of oxides, hydroxides, carbonates and hydrogencarbonates of a metal, for example, a basic inorganic metal compounds. The basic metal compound is preferably a different compound from the aforementioned inorganic compound base material. The above metal is preferably at least one element selected from alkaline earth metal elements, transition metal elements and zinc group elements. Preferred examples of the basic metal compounds include zinc oxide, magnesium hydroxide, magnesium carbonate, calcium carbonate, zinc carbonate and goethite. Of these, preferable is zinc compounds, more preferable is zinc oxide or zinc carbonate, and particularly preferable is zinc oxide. The content of the basic metal compound is preferably 1% by weight to 50% by weight, more preferably 5% by weight to 40% by weight, and particularly preferably 10% by weight to 35% by weight, for example, 15% by weight to 25% by weight, based on the total weight of the removing agent.

As previously described, the removing agent of the present invention comprises a color indicator. The color indicator is not particularly limited, and for example, a pH indicator (acid-base indicator) or an oxidation-reduction indicator can be used. The color indicator is preferably a pH indicator. As the pH indicator in the present invention, any compound is employable as long as it indicates pH through color development, that is, the color thereof changes as the pH of the removing agent changes, and as an example thereof, a pH indicator described in JP2001033438A is preferably used. Examples of pH indicators that can be used include indigo carmine, 1,3,5-trinitrobenzene, nitramine, tropaeolin O, poirrier blue C4B, alizarin yellow GG, alizarin yellow R, thymolphthalein complexon, thymol blue, α-naphtholbenzein, α-cresolphthalein, p-xylenol blue, metacresol purple, α-naphtholphthalein, cyanine, rosolic acid, neutral red, phenolsulfonephthalein, bromocresol purple, methylthymol blue, α-nitrophenol, m-nitrophenol, chlorophenol red, methyl red, bromocresol green, bromophenol blue, methyl orange, bromothymol blue, thymolphthalein, metacresol purple, cresol red, bromophenol red, phenolphthalein and p-nitrophenol. Of these, in the present invention, at least one, or if necessary two or more, selected from the group consisting of bromophenol blue, methyl orange, bromothymol blue, thymolphthalein, metacresol purple, cresol red, bromophenol red, phenolphthalein and p-nitrophenol are preferably used.

In a preferred embodiment of the present invention, the pH indicator is a pH indicator having a transition range in a pH range of 2 to 9. More preferably, the pH indicator is a pH indicator having a transition range in a pH range of 3 to 8. Here, the transition range refers to a range of pH where the addition of the indicator results in the change of color.

In a more preferred embodiment of the present invention, the pH indicator is selected from the group consisting of bromophenol blue, methyl orange and bromothymol blue.

The content of the indicator such as the pH indicator is preferably 0.001 to 5% by weight, more preferably 0.005 to 1% by weight, still more preferably 0.007 to 0.6% by weight, and particularly preferably 0.01 to 0.5% by weight, for example, 0.05 to 0.4% by weight, based on the total weight of the removing agent.

As described above, the chlorine gas removing agent according to the present invention comprises the inorganic compound base material, the sulfur-containing reducing compound and the color indicator such as a pH indicator. As described above, the chlorine gas removing agent according to the present invention can further comprise zinc oxide or another basic metal compound. In addition, the removing agent according to the present invention may comprise other components such as a dispersion medium and a molding aid, within limits not detrimental to the effects of the present invention.

In an embodiment of the present invention, the removing agent substantially consists of only the inorganic compound base material, the sulfur-containing reducing compound, the color indicator and optionally a dispersion medium, or substantially consists of only the inorganic compound base material, the sulfur-containing reducing compound, the color indicator, the basic metal compound and optionally a dispersion medium.

In an embodiment of the present invention, the total weight of the inorganic compound base material, the sulfur-containing reducing compound and the color indicator (or the total weight of the inorganic compound base material, the sulfur-containing reducing compound, the color indicator and the basic metal compound when the removing agent comprises the basic metal compound) in the removing agent can be 70 to 100% by weight, preferably 80 to 100% by weight, and particularly preferably 90 to 100% by weight, for example, 95 to 100% by weight, based on the total weight of the removing agent.

For example, the removing agent according to the present invention comprises the pH indicator, the inorganic compound base material, the sulfur-containing reducing compound and the zinc compound, and the total weight of these components is 90 to 100% by weight based on the total weight of the removing agent.

In an embodiment of the present invention, the compositional ratio by weight among the color indicator, the inorganic compound base material and the sulfur-containing reducing compound in the removing agent can be in the range of, for example, 0.001 to 1.0:30.00 to 97.00:1.00 to 40.00 when the total weight of these components is 100.

In a further embodiment of the present invention, the compositional ratio by weight among the color indicator, the inorganic compound base material, the sulfur-containing reducing compound and the basic metal compound (optional) in the removing agent of the present invention can be in the range of 0.001 to 1.0:30.00 to 97.00:1.00 to 40.00:0.00 to 40.00 when the total weight of these components is 100.

In a more preferred formulation of the removing agent according to the present invention, the compositional ratio by weight among the color indicator, the inorganic compound base material, the sulfur-containing reducing compound and the basic metal compound is in the range of 0.05 to 0.5:50.00 to 80.00:10.00 to 30.00:10.00 to 30.00 when the total weight of these components is 100, and more preferably, the compositional ratio by weight among them is in the range of 0.05 to 0.5:50.00 to 75.00:10.00 to 30.00:10.00 to 30.00 when the total weight of these components is 100.

In an embodiment of the present invention, the removing agent may have a tap density of 0.50 g/ml to 1.50 g/ml, and preferably 0.65 g/ml to 1.30 g/ml, for example, 0.75 g/ml to 1.15 g/ml.

A process for producing the removing agent according to the present invention, uses of the removing agent and a system using the removing agent, etc. are described below. As the inorganic compound base materials, such a large number of materials as previously described can be used, but for a brief description, embodiments using pseudoboehmite as the base material are mainly described herein, and likewise, embodiments using a pH indicator as the color indicator and using zinc oxide as the basic metal compound is mainly described herein. These descriptions are applicable to other embodiments in which an inorganic compound base material other than pseudoboehmite, a color indicator other than the pH indicator and a basic metal compound other than zinc oxide are used, and a person skilled in the art will properly understand other embodiments by referring to these descriptions. Herein, the basic metal compound such as zinc oxide can be optionally used.

The removing agent according to the present invention can be produced by, for example, a method in which pseudoboehmite, the sulfur-containing reducing compound, the pH indicator and zinc oxide, and a dispersion medium that is added if necessary, are mixed/kneaded, then shaped and thereafter dried. The pseudoboehmite, the sulfur-containing reducing compound such as a thiosulfate, and zinc oxide are each usually provided as a powder. In this case, those powders are weighed and mixed. For example, in order to prepare general extruded cylindric pellets, pseudoboehmite, the sulfur-containing reducing compound powder, zinc oxide and the pH indicator can be sufficiently dry-mixed in predetermined amounts in a mixing kneader, and then kneaded after water is added in an amount of 0.1 to 1 part by weight, preferably 0.3 to 0.5 part by weight, based on 1 part by weight of the mixed powder. In this case, the water is desirably divided and introduced so that the mixture should not become heterogeneous. For the kneading, a kneader for food production, such as a grinding machine, can be used. The dispersion medium can be used for the purpose of dispersing pseudoboehmite, the sulfur-containing reducing compound and zinc oxide to homogeneously mix them and for the purpose of imparting a cohesive force for maintaining a fixed shape during the shaping and drying steps. As the dispersion medium, water is preferably used, but organic solvents such as alcohols or other additives can also be used, when needed.

The kneaded raw materials can be then shaped. If the materials in the form of powders are used as they are, the resulting removing agent becomes pasty because of water generated with the decomposition of chlorine gas, and it may become difficult to treat chlorine gas with such a removing agent. In order to prevent the removing agent from losing its shape due to water, etc. generated with the decomposition of chlorine gas, while keeping contact of chlorine gas with the removing agent constant, it is preferable to impart proper mechanical strength and shape to the removing agent.

The shape and size of the removing agent according to the present invention can be appropriately selected depending on the usage form, but in general, a particulate shaped body or a cylindric pellet having a diameter of about 1 to 6 mm and a length of about 3 to 20 mm is preferably used. However, as a matter of course, the shape and size are not limited thereto, and various irregular-shaped pellets, tablets, granulates, crushed granulates and fine particles obtained by spray drying, etc. are employable.

In the present invention, the pore volume of the removing agent can also play an important role, and therefore, a shaping method capable of applying a proper mechanical pressure is preferably used. It is preferable to carry out shaping while applying a pressure of 30 to 200 kg/cm², and particularly preferably a pressure of 50 to 100 kg/cm². As machines for such shaping, general granulators, etc. can be used. Of these, a disc pelleter and a plunger extruder that are capable of adjustment to the above pressure and provide shaped bodies with excellent uniformity are preferably used, and of these, a plunger extruder is particularly preferable.

The shaped removing agent can be then dried. In the present invention, the reducing agent is preferably contained in the form of hydrate thereof in the removing agent, and therefore, the drying temperature is preferably lower than the elimination temperature of the water of hydration. For example, when a thiosulfate is used as the reducing agent, the drying temperature is preferably room temperature to 150° C., more preferably 30 to 140° C., still more preferably 40 to 130° C., and particularly preferably 50 to 120° C., for example, 60 to 115° C. In the case of sodium thiosulfate pentahydrate, however, it is thought that elimination of the water of hydration rapidly proceeds at 60 to 200° C. Accordingly, in a preferred embodiment of the present invention, the drying temperature can also be room temperature to 95° C., preferably 30 to 90° C., more preferably 35 to 80° C., and particularly preferably 40 to 70° C., for example, 40 to 55° C., from the viewpoint of maximum maintenance of water of hydration. The drying time is preferably 10 minutes to one month, more preferably one hour to one week, and particularly preferably 3 hours to 2 days. If the time is too short, the physical strength and the gas removing performance of the removing agent are liable to decrease due to the residual moisture content, etc., and if the time is too long, the efficiency for manufacturing the removing agent is liable to decrease. The drying can be carried out by using, for example, an electric heater, and thereafter, the removing agent can be stored in a container containing a desiccant, when needed.

In an embodiment of the present invention, the present invention relates to a method for producing the halogen gas removing agent, comprising mixing and/or kneading the inorganic compound base material, the sulfur-containing reducing compound, the color indicator and optionally the basic metal compound (for example, pseudoboehmite, a sulfur-containing reducing compound, a pH indicator and optionally zinc oxide) optionally together with a dispersion medium, and then shaping the mixture, followed by drying.

In another embodiment of the present invention, the present invention relates to a halogen gas removing agent produced by a process comprising mixing and/or kneading the inorganic compound base material, the sulfur-containing reducing compound, the color indicator and optionally the basic metal compound (for example, pseudoboehmite, a sulfur-containing reducing compound, a pH indicator and optionally zinc oxide) optionally together with a dispersion medium, and then shaping the mixture, followed by drying. Here, the drying can be carried out at a temperature of, for example, 30 to 140° C., preferably 50 to 120° C., for a period of, for example, 10 minutes to one month, preferably one hour to one week, more preferably 3 hours to 2 days. The shaping can be carried out using, for example, a disc pelleter or a plunger extruder, preferably a plunger extruder.

When chlorine gas is introduced into the removing agent obtained by using the above raw materials, formulation and a production method as above, the reduction/decomposition of chlorine gas occurs, and as a result, passing of chlorine gas through the removing agent is inhibited, and besides, hydrogen chloride formed is also trapped in the removing agent. The chemical reactions during this time are represented by the formulae (1) to (6).

In the case where the removing agent does not comprise the basic metal compound such as zinc oxide, hydrogen chloride HCl is generated by the sulfur-containing reducing compound, the hydrogen chloride HCl further reacts with the sulfur compound to convert into a chlorine compound, and the chlorine compound is trapped in the removing agent. If the activity of the sulfur-containing reducing compound is low or the amount of said compound added is small, the rate of diffusion of the chlorine gas becomes higher than the rate of decomposition thereof, and the chlorine gas diffuses in the removing agent and breaks through the outlet.

(Case where the Removing Agent does not Comprise the Basic Metal Compound)

4Cl₂+Na₂S₂O₃·5H₂O→6HCl+2H₂SO₄+2NaCl Formula (1)

Na₂S₂O₃·5H₂O+2HCl→SO₂+S+2NaCl+6H₂O Formula (2)

Na₂S₂O₃·5H₂O+H₂SO₄→SO₂+S+Na₂SO₄+6H₂O Formula (3)

### (Case Where the Removing Agent Comprises Basic Metal Compound: Example Using ZnO)

4Cl₂+Na₂S₂O₃·5H₂O→6HCl+2H₂SO₄+2NaCl Formula (4)

ZnO+2HCl→ZnCl₂+H₂O Formula (5)

ZnO+H₂SO₄→ZnSO₄+H₂O Formula (6)

When the basic metal compound such as zinc oxide is added to the removing agent, the chemical reaction formulae are represented by (4) to (6). The decomposition of chlorine is accelerated by the decomposition action of the sulfur-containing reducing compound and, by way of hydrogen chloride, solid zinc chloride is fixed in the removing agent. As a result, the contribution of the reactions represented by formulae (2) and (3) decreases and, as can be seen from a comparison between Example 2 and Example 4 described later, if the basic metal compound is added to the removing agent, the breakthrough time of sulfurous acid gas markedly increases and comes close to the breakthrough time of hydrogen chloride. From this, it can be said that, by selecting and controlling the type and the amount of the basic metal, it also becomes possible to select a gas that breaks through earlier.

As one definition of a life of the chlorine removing agent, a breakthrough time of any one of chlorine gas, hydrogen chloride gas and sulfurous acid gas can be mentioned. If there is an indicator that can react with any of these gases and exhibit color, diffusion of the gas in the removing agent can be detected with such an indicator, and by monitoring said color, the life of the removing agent can be predicted, and also the breakthrough time can be measured. If an oxidation-reduction indicator is used as the color indicator, the indicator develops color due to oxidation/reduction action of chlorine gas or sulfurous acid gas, so that diffusion of the gas can be detected. If a pH indictor is used, any one of chlorine gas, hydrogen chloride gas and sulfurous acid gas can be detected. In the present invention, it is preferable to use a pH indicator as the detection agent.

As previously described, if the gas that breaks through first is chlorine gas having strong toxicity, the influence that is exerted when the gas leaks and diffuses outside is much larger than that of hydrogen chloride or sulfurous acid gas. On that account, from the viewpoint of safety, it is preferable that prior to breakthrough of chlorine gas, breakthrough of hydrogen chloride gas or sulfurous acid gas take place and the removing agent reaches the end of its life, thereby being replaced. That is to say, the diffusion rate of hydrogen chloride gas in the removing agent is preferably higher than the diffusion rate of chlorine gas.

When a sufficient amount of zinc oxide is contained in the removing agent, it will be fixed therein in the form of non-volatile zinc chloride or zinc sulfate, as shown by the formulae (4) to (6), and therefore leakage of a harmful gas is prevented. If zinc oxide is used up, hydrogen chloride may diffuse in and break through the removing agent. Thus, in the removing agent according to the present invention, controlling the amounts of the reducing agent and the basic metal compound such as zinc oxide can enhance the decomposition of chlorine gas and can delay the breakthrough time of chlorine gas and, in addition, monitoring the color change of the pH indicator enables the prediction of the life of the removing agent. Contrary to this, if the compositional ratio of the pseudoboehmite, the sulfur-containing reducing compound and zinc oxide is not appropriate, it may not be noticed that the color change of the pH indicator is caused by chlorine even if the color change of the pH indicator accompanying diffusion of chlorine gas can be monitored, and the problem of leakage of chlorine gas may occur. In order to avoid such a problem, the compositional ratio by weight among the pH indicator, pseudoboehmite, the sulfur-containing reducing compound and the zinc compound is preferably 0.05 to 0.5:50.00 to 80.00:10.00 to 30.00:10.00 to 30.00, for example, 0.05 to 0.5:50.00 to 75.00:10.00 to 30.00:10.00 to 30.00, when the total weight of these components is 100, and this corresponds to the weight ratio described previously as a preferred compositional ratio by weight among the color indicator, the inorganic compound base material, the sulfur-containing reducing compound and the basic metal compound.

A system for removing chlorine gas using the removing agent of the present invention is schematically shown in FIG. 1. Chlorine gas exhausted from a chlorine gas emission source, e.g. a semiconductor production apparatus such as a dry etching apparatus, flows into a column packed with the chlorine removing agent, then the gas is decomposed, fixed and purified as previously described, and a detoxified gas (that is, gas from which chlorine gas has been removed) such as water is exhausted. When sulfurous acid gas or hydrogen chloride is formed as a result of inflow of the chlorine gas, color change of the removing agent begins from the inlet side of the chlorine gas removing column and the color-changed region spreads to the outlet side with time. By visually observing the appearance of this color change through a graduated transparent window or by measuring the length of the discolored potion, the residual ability of the removing agent can be predicted. If necessary, for example, a color detection device comprising light emission diode and an optical sensor in combination can be provided, whereby automatic monitoring of the state of consumption of the removing agent also becomes possible. If providing a breakthrough detection sensor as a stage following the removing agent column, it is also possible to give an alarm when hydrogen chloride or sulfurous acid gas is detected by the sensor, and by virtue of this, even if these gases break through from the removing agent column, leakage of chlorine gas can be prevented by stopping the operation of the apparatus, so that safety can be further enhanced.

Methods of using the removing agent of the present invention are not particularly limited, and for example, the removing agent can also be used in a moving bed or a fluidized bed, but it is usually used in a fixed bed. For example, a tubular column is packed with the removing agent, and a chlorine gas-containing gas is introduced into the column, whereby chlorine gas can be removed safely and efficiently. Such removal of chlorine gas can be carried out for exhaust gas containing chlorine gas of, for example, 0.01 ppmv to 100% by volume, preferably 0.1 ppmv to 10% by volume, more preferably 1 ppmv to 5% by volume; and/or can be carried out at a temperature of 200° C. or lower, preferably 10 to 100° C., more preferably 20 to 90° C., for example, room temperature; and/or can be carried out with a removing agent bed thickness of 1 to 1000 cm, for example, 10 cm to 200 cm; and/or can be carried out at a chlorine-containing gas space velocity of 1 to 2000 h⁻¹, for example, 100 to 1000 h⁻¹.

As described above, the function of the pH indicator in the removing agent is to detect hydrogen chloride gas. However, as shown by the formulae (2) and (3), when the basic metal compound such as zinc oxide is not present in the removing agent, sulfurous acid gas is generated. This sulfurous acid gas is partially fixed by pseudoboehmite, but if the amount of the sulfurous acid gas exceeds the fixing ability of the pseudoboehmite, said gas breaks through the removing agent thereby causing environmental pollution, similarly to hydrogen chloride. It is also possible to give a role of detecting this acidic sulfurous acid gas to the pH indicator of the present invention. In this case, by using two or more different pH indicators, for example, by using a pH indicator that changes the color thereof if detecting hydrogen chloride and a pH indicator that changes the color thereof if detecting sulfurous acid gas, the former being a different indicator from the latter, diffusion of each gas in the column can be observed.

In an embodiment of the present invention, the present invention relates to a halogen gas removing apparatus, comprising a container, and a window and/or a color sensor, said window and/or said color sensor being provided in the container, wherein
the container comprises a gas flow inlet and a gas flow outlet,
the container comprises the removing agent packed in said container, and
the window and/or the color sensor are adapted for observation and/or detection of the color change of the removing agent accompanying the removal of the halogen gas. From the gas flow inlet, the halogen gas-containing gas (the halogen gas in a gas flow) is introduced.

In another embodiment of the present invention, the present invention relates to a method for monitoring the state of consumption of the halogen gas removing agent, using the above apparatus, by measuring the length of a color-changed portion in the removing agent from the halogen gas inflow end. Specifically, for example, when a halogen-containing gas is introduced to the removing agent (bed) packed in a container having a gas flow inlet and a gas flow outlet to perform removal of the halogen gas, the removing agent changes the color thereof as it is consumed, as described above, and the color-changed region begins from the halogen gas inflow end (the end on the gas flow inlet side) of the removing agent zone (removing agent bed) and extends in the direction of the outflow end (the end on the gas flow outlet side) thereof. Therefore, by arranging the aforementioned window and/or color sensor in the container, and measuring the size of the color-changed region in the removing agent zone (removing agent bed) or, for convenience, measuring the length between the halogen gas inflow end of the removing agent zone (removing agent bed) and the color-changed area/non-color-changed area boundary, by means of the window and/or the color sensor, the state of color change of the removing agent can be continuously observed and/or detected, whereby the state of consumption of the removing agent (that is, how much the removing agent is saturated with the halogen-based gas relative to the limit of the gas fixing ability of the removing agent) can be monitored.

The present invention further relates to a method for removing a halogen gas from a halogen-containing gas, comprising bringing the halogen-containing gas into contact with the removing agent. In a further embodiment of the present invention, the present invention relates to a method for removing a halogen gas from a halogen-containing gas, comprising bringing the halogen-containing gas into contact with the removing agent, wherein the halogen gas is removed while the state of consumption of the removing agent is monitored by observing and/or detecting the color change of the removing agent accompanying the removal of the halogen gas. In these embodiments, the aforesaid apparatus can be preferably used. Moreover, for the monitoring, the aforesaid monitoring method can also be used. For example, the above contact can be carried out for a halogen-containing gas containing a halogen gas of 0.01 ppmv to 100% by volume, preferably 0.1 ppmv to 10% by volume, more preferably 1 ppmv to 5% by volume; and/or can be carried out at a temperature of 200° C. or lower, preferably 10 to 100° C., more preferably 20 to 90° C., for example, room temperature; and/or can be carried out with a removing agent bed thickness of 1 to 1000 cm, for example, 10 cm to 200 cm; and/or can be carried out at a halogen-containing gas space velocity of 1 to 2000 h⁻¹, for example, 100 to 1000 h⁻¹.

In an embodiment of the present invention, the present invention relates to use of the above apparatus for monitoring the state of consumption of the halogen gas removing agent by measuring the length of the color-changed portion in the removing agent from the halogen gas inflow end.

In a further embodiment of the present invention, the present invention relates to a use of the above removing agent for removing a halogen gas from a gas containing a halogen gas under the following conditions, or use of the above removing agent for removing a halogen gas from a halogen-containing gas under the following conditions, wherein the halogen gas is removed while the state of consumption of the removing agent is monitored by observing and/or detecting the color change of the removing agent accompanying the removal of the halogen gas:
halogen gas concentration in the halogen gas-containing gas: 0.01 ppmv to 100% by volume; and/or
temperature: 200° C. or lower; and/or
removing agent bed thickness: 1 to 1000 cm; and/or
space velocity of the halogen gas-containing gas: 100 to 1000 h⁻¹.

The present invention is described below in more detail with reference to the following examples, but the present invention is in no way limited to those examples.

### EXAMPLES

Evaluation of characteristics and evaluation of performance of removing agents used in the following examples and comparative examples were carried out by the methods described below.

(1) Reflectance measurement of samples: Using Model V-650 from JASCO Corporation and an integrating sphere unit (Model ISV-722 from JASCO Corporation), a standard white plate (Spectralon™ from Labsphere Inc., USA) was subjected to measurement of an ultraviolet visible diffuse reflection spectrum. Thereafter, removing agent samples were subjected to measurement of an ultraviolet visible diffuse reflection spectrum in the same manner as above. From the results, a relative diffuse reflectance R_{∞} was calculated using the formula (I).

R_{∞}=R_{∞}^{s}/R_{∞}⁰ Formula (I)
R_{∞}^{s}: diffuse reflection spectrum of sample
R_{∞}⁰: diffuse reflection spectrum of standard white plate

The spectral intensity is expressed by a Kubelka-Munk function F(R_{∞}) from the relative diffuse reflectance R_{∞} using the following formula (II).

F(R_{∞})=(1−R_{∞})²/2R_{∞} Formula (II)

(2) Color tone evaluation test: a jacketed transparent glass tubular reactor having an inner diameter of 2.23 cm was packed with 20 ml of a removing agent, then dry nitrogen containing 1.0% by volume of chlorine (Cl₂) gas was introduced into the reactor at a space velocity (GHSV) of 500 h⁻¹ for 12 hours or more using a mass flow controller, thereafter a 5 ml sample of the removing agent at the gas inlet in the reactor was taken, and said sample was subjected to the reflectance measurement and color change observation.

(3) Tap density measurement of removing agent: 100 g of a removing agent was placed in a 200 ml graduated cylinder, and after tapping was carried out 100 times, the volume was read out, whereby the tap density (g/ml) was examined. Autotap from Quantachrome Instruments Japan was used as the measurement apparatus.

(4) Evaluation of chlorine removing ability: a jacketed transparent glass tubular reactor having an inner diameter of 2.23 cm was packed with 20 ml of a removing agent that was a test object, then dry nitrogen containing 1.0% by volume of chlorine (Cl₂) gas was introduced into the reactor at a space velocity (GHSV) of 500 h⁻¹ using a mass flow controller, and a gas introducing time until detection of 1 ppmv of chlorine gas, hydrogen chloride (HCl) gas or sulfurous acid gas in the gas to be treated was examined. Temperature control was carried out by circulating constant-temperature water into the jacket, and the temperature was set at 25° C. or 80° C. For the detection of chlorine gas, a detector tube (No 8La) from Gastec Corporation was used, for the detection of hydrogen chloride gas, a detector tube (No 14L) from Gastec Corporation was used, and analysis was carried out every 10 min to 15 min. The chlorine removing ability (L/kg) of the treating agent was calculated using the following formula (III).

Chlorine removing ability (L/kg)=space velocity (500 h⁻¹)×chlorine concentration (1.0% by volume)×time during which chlorine gas is treated (h)/tap density (g/ml) Formula (III)

(5) Detection of sulfurous acid gas: For the detection of sulfurous acid gas (SO₂), a detector tube (No 5La) from Gastec Corporation was used.

(6) Monitoring of state of consumption of removing agent: in the above test (2) or (4), a change in hue of the removing agent over time was observed through a glass tubular reactor.

### Example 1

The process for preparing a removing agent sample was as follows. A bromophenol blue powder, a pseudoboehmite powder (specific surface area: 340 m²/g) and a sodium thiosulfate pentahydrate powder were weighed in such a manner that the amounts of bromophenol blue, pseudoboehmite and sodium thiosulfate pentahydrate became 0.01% by weight, 81.99% by weight and 18.00% by weight, respectively, and they were mixed using a grinding machine (manufactured by Ishikawa Kojo Co. Ltd., Model 18) while water was added thereto, whereby a kneaded cake was obtained. Using a plunger extruder, the kneaded cake was shaped into a particulate shaped body having a diameter of about 2 mm and a length of about 6 mm. The resulting shaped body was dried overnight in an electric dryer kept at 110° C., thereafter placed in a desiccator and held for one hour or more to decrease the temperature to room temperature, whereby a removing agent sample of Example 1 was obtained. The resulting sample was subjected to the color tone evaluation test. The color tone of the removing agent changed from blue before the treatment of chlorine to yellow after the treatment.

### Example 2

A removing agent sample of Example 2 (tap density: 0.85 g/ml) comprising 0.01% by weight of bromothymol blue, 81.99% by weight of pseudoboehmite and 18.00% by weight of sodium thiosulfate pentahydrate was prepared in an analogous manner under the same conditions as in Example 1. The resulting sample was subjected to the chlorine removing evaluation at 25° C. After inflow of chlorine gas began, color change of the removing agent gradually began from the vicinity of the inlet, and a phenomenon that the color-changed region increased over time was observed. At 150 minutes after starting the introduction of chlorine gas, sulfurous acid gas was detected first, and at the same time, the color-changed region reached the outlet. At 240 minutes, hydrogen chloride gas was detected. When the first gas breakthrough time was defined as the ability of a removing agent, the ability of this removing agent was 14 Lkg⁻¹. In addition, the sample obtained by the above preparation was subjected to the color tone evaluation test. The color tone of the removing agent changed from blue before the color tone evaluation test to red after the test. The reason why the color tone changed to red after the evaluation is thought to be that the pH of the sample was much lower than pH 6.0 at which bromothymol blue changes to yellow. This can be applied to other examples.

### Example 3

A removing agent sample of Example 3 comprising 0.01% by weight of phenolphthalein, 81.99% by weight of pseudoboehmite and 18.00% by weight of sodium thiosulfate pentahydrate was prepared in an analogous manner under the same conditions as in Example 1. The resulting sample was subjected to the color tone evaluation test. The color tone of the removing agent changed from red before the color tone evaluation test to white after the test.

### Example 4

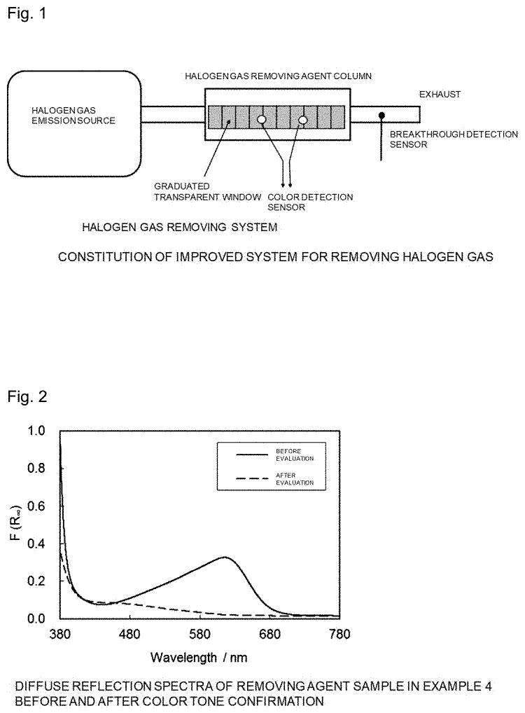

The process for preparing a removing agent sample was as follows. A bromothymol blue powder, a pseudoboehmite powder, a sodium thiosulfate pentahydrate powder and a zinc oxide powder were weighed in such a manner that the percentage composition by weight of the bromothymol blue powder, the pseudoboehmite powder, the sodium thiosulfate pentahydrate powder and the zinc oxide powder became 0.01%, 59.99%, 20.00% and 20.00%, respectively, and subsequently, a sample of Example 4 (tap density: 1.06 g/ml) was obtained in the same manner as in Example 1. The resulting sample was subjected to the evaluation of chlorine removing ability at 25° C. After inflow of chlorine gas began, color change of the removing agent gradually began from the vicinity of the inlet, and a phenomenon that the color-changed region increased over time was observed. At 400 minutes after starting the introduction of chlorine gas, sulfurous acid gas was detected first, and at the same time, the color-changed region reached the outlet. At 460 minutes, hydrogen chloride gas was detected. When the first gas breakthrough time was defined as the ability of a removing agent, the ability of this removing agent was 30 Lkg⁻¹. Before and after the evaluation of chlorine removing ability, the sample was subjected to the reflectance measurement. The results are shown in FIG. 2. The color tone changed from blue before the evaluation to red after the evaluation.

### Example 5

The removing agent sample prepared in Example 4 was subjected to the color tone evaluation test at 80° C. The color tone of the removing agent changed from blue before the color tone evaluation test to red after the test.

### Example 6

A removing agent sample of Example 6 was prepared in the same manner under the same conditions as in Example 4, except that the bromothymol blue, the pseudoboehmite, the sodium thiosulfate pentahydrate and the zinc oxide were weighed in such a manner that the percentage composition by respective weights thereof were 0.30%, 59.70%, 20.00% and 20.00%. The resulting sample was subjected to the color tone evaluation test. The color tone of the removing agent changed from blue before the color tone evaluation test to red after the test.

### Comparative Example 1

The process for preparing a sample of Comparative Example 1 was as follows. A pseudoboehmite powder and a sodium thiosulfate pentahydrate powder were weighed in such a manner that the percentage composition by weight of the pseudoboehmite powder and the sodium thiosulfate pentahydrate powder was 82.00% and 18.00%, respectively. A sample of Comparative Example 1 was prepared in the same manner as in Example 1, except that the pH indicator was not added. The resulting sample was subjected to the evaluation of the removing ability at 25° C. As a result, at 60 minutes after starting the introduction of chlorine gas, sulfurous acid gas was detected, and at 240 minutes, hydrogen chloride gas was detected. When the first gas breakthrough time was defined as the ability of a removing agent, the ability of this removing agent was 5 Lkg⁻¹. The sample obtained by the above preparation was subjected to the color tone evaluation test. The color tone of the removing agent was still white even after the color tone evaluation test, and the difference in F(R_{∞}) between the samples before and after the introduction of gas was small, in the measurement of sample reflectance.

### Comparative Example 2

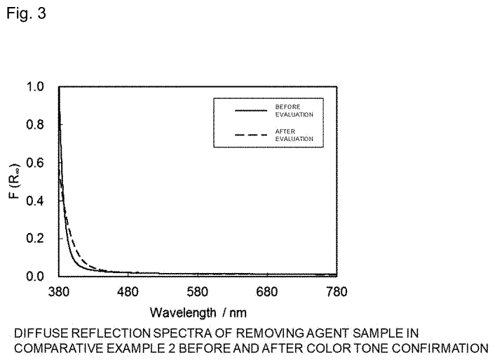

The process for preparing a sample of Comparative Example 2 was as follows. A pseudoboehmite powder, a sodium thiosulfate pentahydrate powder and a zinc oxide powder were weighed in such a manner that the percentage composition by weight of the pseudoboehmite powder, the sodium thiosulfate pentahydrate powder and the zinc oxide powder was 60.00%, 20.00% and 20.00%, respectively. A sample of Comparative Example 2 was prepared in the same manner as in Example 4, except that the pH indicator was not added. The resulting sample was subjected to the evaluation of chlorine removing ability at 25° C. As a result, at 420 minutes after starting the introduction of chlorine gas, sulfurous acid gas was detected, and at 450 minutes, hydrogen chloride gas was detected. When the first gas breakthrough time was defined as the ability of a removing agent, the ability of this removing agent was 34 Lkg⁻¹. Before and after the evaluation of chlorine removing ability, the sample was subjected to the reflectance measurement. The results are shown in FIG. 3, but the color change was extremely small and difficult to observe visually.

The evaluation results of the samples of the examples and the comparative examples are set forth in Table 1.

TABLE 1
Evaluation results of chlorine removing agent

| | | | | Difference in | | Sulfurous | |
|---|---|---|---|---|---|---|---|
| | | Temperature for | | F(R_{∞}) between | Hydrogen | acid | Removing |
| | | evaluation of | Reflectance | before and after | chloride | gas | agent |
| | | chlorine removing | peak | introduction of | breakthrough | breakthrough | ability |
| | Raw material mixing ratio | ability | wavelength | gas | time | time | (Lkg⁻¹) |
| Ex. 1 | 0.01 wt % Bromophenol blue | 25° C. | 597 nm | 0.150 | — | — | — |
| | (Transition range pH: 3.0 to 4.6) | | | | | | |
| | 81.99 wt % Pseudoboehmite | | | | | | |
| | 18.00 wt % Sodium thiosulfate pentahydrate | | | | | | |
| Ex. 2 | 0.01 wt % Bromothymol blue | 25° C. | 617 nm | 0.091 | 240 min | 150 min | 14 |
| | (Transition range pH: 6.0 to 7.6) | | | | | | |
| | 81.99 wt % Pseudoboehmite | | | | | | |
| | 18.00 wt % Sodium thiosulfate pentahydrate | | | | | | |
| Ex. 3 | 0.01 wt % Phenolphthalein | 25° C. | 537 nm | 0.010 | — | — | — |
| | (Transition range pH: 8.3 to 10.0) | | | | | | |
| | 81.99 wt % Pseudoboehmite | | | | | | |
| | 18.00 wt % Sodium thiosulfate pentahydrate | | | | | | |
| Ex. 4 | 0.01 wt % Bromothymol blue | 25° C. | 617 nm | 0.306 | 460 min | 400 min | 30 |
| | 59.99 wt % Pseudoboehmite | | | | | | |
| | 20.00 wt % Sodium thiosulfate pentahydrate | | | | | | |
| | 20.00 wt % zinc oxide | | | | | | |
| Ex. 5 | 0.01 wt % Bromothymol blue | 80° C. | 617 nm | 0.289 | — | — | — |
| | 59.99 wt % Pseudoboehmite | | | | | | |
| | 20.00 wt % Sodium thiosulfate pentahydrate | | | | | | |
| | 20.00 wt % zinc oxide | | | | | | |
| Ex. 6 | 0.30 wt % Bromothymol blue | 25° C. | 617 nm | 1.628 | — | — | — |
| | 59.70 wt % Pseudoboehmite | | | | | | |
| | 20.00 wt % Sodium thiosulfate pentahydrate | | | | | | |
| | 20.00 wt % zinc oxide | | | | | | |
| Comp. Ex. 1 | 82.00 wt % Pseudoboehmite | 25° C. | 423 nm | 0.001 | 240 min | 60 min | 5 |
| | 18.00 wt % Sodium thiosulfate pentahydrate | | | | | | |
| Comp. Ex. 2 | 60.00 wt % Pseudoboehmite | 25° C. | 642 nm | 0.002 | 450 min | 420 min | 34 |
| | 20.00 wt % Sodium thiosulfate pentahydrate | | | | | | |
| | 20.00 wt % zinc oxide | | | | | | |

The above results can be sorted out as follows.

1) It can be seen from Example 2 that, when a removing agent consisting of pseudoboehmite, sodium thiosulfate and a pH indicator was used for the treatment of chlorine gas, sulfurous acid gas broke through first and hydrogen chloride gas broke through next. Almost simultaneously with the arrival of the color-changed region at the outlet, the detector detected sulfurous acid gas. The reason for the color change is presumed to be that, since the removing agent is neutral to weakly alkaline, the pH thereof is shifted to the acidic side by the generation of sulfurous acid gas, whereby the removing agent changes its color.

2) From the results of Example 1 (transition range of pH indicator bromophenol blue: pH 3.0 to 4.6), Example 2 (transition range of pH indicator bromothymol blue: pH 6.0 to 7.6) and Example 3 (transition range of pH indicator phenolphthalein: pH 8.3 to 10.0), it is preferable to use a pH indicator having a transition range in the pH range of 2 to 9, preferably 3 to 8.

3) Comparing Example 2 and Example 4, the breakthrough times of both of sulfurous acid gas and hydrogen chloride became longer and the delayed time in the case of hydrogen chloride was reduced, by the addition of zinc oxide.

4) It can be understood that sulfurous acid, hydrogen chloride or zinc chloride that was a reaction product of zinc oxide with hydrogen chloride contributed to the color change reaction in Example 4.

5) When replacing a removing agent by a new one, the time required for the color change reaches the outlet can be regarded as an indication of the life of a removing agent. Related to this, the addition of zinc oxide to the removing agent raised the chlorine removing ability thereof by about 2.1 times. As shown in Table 1, moreover, by the addition of zinc oxide, the hue of the removing agent before use became stronger, and therefore, the degree of color change accompanying the treatment of chlorine gas greatly increased, thereby enhancing visibility during monitoring by an operator.

6) As shown in Table 1, Example 6, in which the amount of the pH indicator was increased as compared with Example 4, shows a difference in the value of F(R.) at 617 nm between before and after the introduction of chlorine gas; in other words, the detection sensitivity, grew about 5.3 times owing to the increase in the amount of the pH indicator. If a window was provided to visually monitor the state of consumption of the removing agent, observation was able to be carried out very easily, and also in the measurement by a color sensor, a signal with high S/N was able to be obtained, resulting in an advantageous measurement.

7) In Example 4 and Example 5, in which color tone evaluation tests were carried out at temperatures of 25° C. and 80° C., respectively, the differences in F(R.) between before and after the introduction of chlorine gas were almost the same. Given that pseudoboehmite, sodium thiosulfate and zinc oxide do not have a thermal decomposition temperature of 200° C. or less, it is also possible to use the chlorine removing agent having a detection function, according to the present invention, at a temperature of around 100° C. or higher.

8) A detector for detecting hydrogen chloride or chlorine gas cannot be used as a detector for sulfurous acid gas, and vice versa; and therefore, when a conventional removing agent having no color function is used, two or three types of detectors for detecting the first gas breakthrough need to be installed to enable detecting whichever gas breaks through first. According to the removing agent of the present invention, the arrival of any gas at the outlet can be recognized as a color change of the removing agent, therefore the safety is further enhanced, and besides, non-use of a detector becomes possible, so that the removing agent of the present invention is preferable also from the viewpoints of cost reduction and safety enhancement.

1. A halogen gas removing agent comprising an inorganic compound, a sulfur-containing reducing compound and a color indicator.

2. The halogen gas removing agent according to claim 1, wherein a halogen gas comprises at least one selected from the group consisting of fluorine (F₂), chlorine (Cl₂), bromine (Br₂) and iodine (I₂).

3. (canceled)

4. (canceled)

5. The halogen gas removing agent according to claim 1, wherein the inorganic compound is selected from the group consisting of metal oxides, metal hydroxides and metal carbonates.

6. The halogen gas removing agent according to claim 1, wherein the inorganic compound comprises an alumina-based compound.

7. The halogen gas removing agent according to claim 1, wherein the inorganic compound comprises pseudoboehmite and/or montmorillonite.

8. The halogen gas removing agent according to claim 1, wherein the inorganic compound has a specific surface area of 100 m²/g to 500 m²/g.

9. The halogen gas removing agent according to claim 1, wherein the inorganic compound has a specific surface area of 200 m²/g to 400 m²/g.

10. The halogen gas removing agent according to claim 1, further comprising a basic metal compound.

11. The halogen gas removing agent according to claim 10, wherein the basic metal compound is at least one zinc compound selected from the group consisting of zinc carbonate and zinc oxide.

12. The halogen gas removing agent according to claim 1, wherein the sulfur-containing reducing compound is at least one compound selected from the group consisting of thiosulfates, sulfites, dithionites and tetrathionates.

13. The halogen gas removing agent according to claim 1, wherein the sulfur-containing reducing compound comprises thiosulfates selected from the group consisting of sodium thiosulfate, potassium thiosulfate and ammonium thiosulfate.

14. The halogen gas removing agent according to claim 1, wherein the sulfur-containing reducing compound comprises hydration water.

15. The halogen gas removing agent according to claim 1, wherein the color indicator is a pH indicator having a transition range in a pH range of 2 to 9.

16. The halogen gas removing agent according to claim 1, wherein the color indicator is a pH indicator having a transition range in a pH range of 3 to 8.

17. The halogen gas removing agent according to claim 1, wherein the color indicator comprises at least one pH indicator selected from the group consisting of bromophenol blue, methyl orange and bromothymol blue.

18. The halogen gas removing agent according to claim 1, wherein a compositional ratio by weight among the color indicator, the inorganic compound and the sulfur-containing reducing compound is 0.001 to 1.0:30.00 to 97.00:1.00 to 40.00 when the total of these components is 100.

19. The halogen gas removing agent according to claim 10, wherein the compositional ratio by weight among the color indicator, the inorganic compound, the sulfur-containing reducing compound and the basic metal compound is 0.05 to 0.5:50.00 to 75.00:10.00 to 30.00:10.00 to 30.00 when the total of these components is 100.

20. The halogen gas removing agent according to claim 19, wherein the total weight of the color indicator, the inorganic compound, the sulfur-containing reducing compound and the basic metal compound is 90 to 100% by weight, based on the total weight of the halogen gas removing agent.

21. A method for producing a halogen gas removing agent comprising steps of:
mixing and/or kneading a color indicator, an inorganic compound and a sulfur-containing reducing compound, together with a dispersion medium, and then shaping the mixture, followed by drying.

22. A halogen gas removing apparatus, comprising a container, and a window and/or a color sensor, said window and/or said color sensor located in the container, wherein
the container comprises a gas flow inlet and a gas flow outlet, wherein
the container comprises the halogen gas removing agent according to claim 1 packed in said container, and
the window and/or the color sensor observe and/or detect a color change of the removing agent accompanying the removal of the halogen gas.

23. A method for monitoring the state of consumption of the halogen gas removing agent, using the apparatus according to claim 22, by measuring the length of a color-changed portion in the halogen gas removing agent, from the halogen gas inflow end of the halogen gas removing agent.

24. A method for removing a halogen gas from a halogen-containing gas, comprising bringing the halogen-containing gas into contact with the removing agent according to claim 1, wherein the halogen gas is removed while the state of consumption of the removing agent is monitored by observing and/or detecting a color change of the removing agent accompanying the removal of the halogen gas.

25. The halogen gas removing agent according to claim 10, wherein a compositional ratio by weight among the color indicator, the inorganic compound, the sulfur-containing reducing compound and the basic metal compound is 0.001 to 1.0:30.00 to 97.00:1.00 to 40.00:10.00 to 40.00 when the total of these components is 100.

26. The method for removing a halogen gas from a halogen-containing gas according to claim 24, wherein the halogen-containing gas is brought from a gas flow from a semiconductor production process.
